(19) United States
(12) Patent Application Publication
(10) Pub. No.: US 2020/0155191 A1
(43) Pub. Date: May 21, 2020

(54) Follicle Punch For Use With Curled Follicles

(71) Applicant: Sanusi Umar, Redondo Beach, CA (US)

(72) Inventor: Sanusi Umar, Redondo Beach, CA (US)

(21) Appl. No.: 16/192,643

(22) Filed: Nov. 15, 2018

(51) Int. Cl. A61B 17/3205

(52) U.S. Cl. CPC A61B 17/32053; A61B 2017/00752

(57) ABSTRACT

A punch that is particularly useful for removing curled hair follicles from a donor site comprises a generally tubular body disposed about a generally longitudinal axis between distal and proximal ends, and having a distal cutting end region terminating distally in an opposing pair of distally-extending, generally prong-like members having an anterior notch and a posterior notch therebetween. In one variation, the prong-like members each have a curved anterior cutting edge and a curved posterior edge meeting at a cutting tip. In the preferred embodiment, the posterior notch extends proximally further than the anterior notch and has a generally “V”-shaped distal segment which carries cutting edges together with a generally “U”-shaped proximal segment extending proximally from the distal segment.

### RELATED APPLICATION

This application is a continuation-in-part of U.S. non-provisional patent application Ser. No. 15/949,357 filed on Apr. 10, 2018 (“the '357 Application”).

The '357 application is a continuation of U.S. non-provisional patent application Ser. No. 14/387,984 filed on Sep. 25, 2014 which issued as U.S. Pat. No. 9,987,038 (“the '038 patent”).

The '038 patent is a nation stage entry of PCT Application US2013/071991 filed on Nov. 26, 2013 (“the '991 application”).

The '991 application claims priority to provisional patent application U.S. Ser. No. 61/874,664 filed on Sep. 6, 2013 and to provisional patent application U.S. Ser. No. 61/729,733 filed on Nov. 26, 2012.

The entire contents of all applications listed in paragraphs 0001-0004 is herein incorporated by reference.

### BACKGROUND

This invention relates to surgical instruments and, more particularly, to a punch for extracting hair follicles from the skin.

Hair transplantation is a surgical technique that involves moving skin containing hair follicles from one part of the body (the donor site) to bald or balding parts (the recipient site).

Hair naturally grows in follicles that contain groupings of 1 to 4 hairs, and transplant techniques typically move the 1-4 hair “follicular units” from the donor site to the recipient site.

The follicles of hair are typically removed from the donor site using punches of between 0.7 mm and 1.25 mm in diameter. The punches are tubular bodies having a skin-contacting cutting edge, and are typically mounted in a tool that causes the punch to rotate as the punch is brought into contact with the donor site. Hair follicles are very easily damage during the removal process, and damaged follicles are unlikely to be successfully transplanted.

Curled follicles are extremely susceptible to damage by follicle punches and are therefore particularly difficult to extract for successful transplantation. Such follicles are curled beneath the skin and are easily cut and/or damaged by the advancing cutting edge of conventional punches as the punch penetrates the donor site's tissue.

### SUMMARY

A punch that is particularly useful for removing curled hair follicles from a donor site comprises a generally tubular body disposed about a generally longitudinal axis and having a distal cutting end region terminating distally in a plurality of distally-extending circumferentially disposed, generally prong-like members carrying distally diverging cutting edges and separated by follicle-accommodating slits. In practice, the punch is oriented during the extraction process at the donor site so that the curled hair root passes into, and is spared from the advancing cutting edge by, a slit as the punch is inserted into and penetrates the tissue. The punch may then rotated slightly so that the cutting edges cut most of the tissue surrounding the follicle without making damaging contact with the follicle. It may be noted that a rotary motion may not be necessary and, if rotation is desired, it may be in one direction or be in the form of an oscillatory rotary movement, depending on characteristics of the donor site and targeted follicle.

The foregoing insertion process may be performed manually or under machine or computer control, and with or without the aid of an ultrasonic transducer coupled to punch to impart a vibratory cutting force against the tissue. In addition, a mechanism for automatically rotating the punch may be employed, and may accordingly be coupled to the ultrasonic transducer if one is used.

These and further details of the invention will be apparent to those of ordinary skill in the art from reading a description of the currently preferred embodiment of the invention described below, of which the drawing forms a part.

### BRIEF DESCRIPTION OF THE FIGURES

The detailed description of some embodiments of the invention is made below with reference to the accompanying figures, wherein like numerals represent corresponding parts of the figures.

FIG. 1 is a front elevation view of a punch for removing hair follicles that is constructed in accordance with the invention;

FIG. 2 is a side elevation view of a punch of FIG. 1;

FIG. 3 is a longitudinal section view of the punch of FIG. 1, taken along line 3-3 in FIG. 1;

FIG. 4 is a longitudinal section view of the punch of FIG. 1, taken along line 4-4 in FIG. 2;

FIG. 5 is an enlarged fragmentary view of the portion of the punch illustrated within the line 5 of FIG. 4;

FIG. 6 is a bottom plan view of the punch of FIG. 2;

FIG. 7 is a fragmentary view in perspective of the cutting end region of the punch oriented per line 7-7 in FIG. 6 illustrated in FIG. 1;

FIG. 8 is an oblique fragmentary elevation view of the cutting end region of the punch of FIG. 1;

FIG. 9 is an oblique bottom view of the cutting end region of the punch of FIG. 1;

FIG. 10 is a schematic illustration of the preferred methodology for extracting a curled follicle in accordance with the invention;

FIG. 11A is a right front oblique view, in schematic, of an alternative embodiment of a punch for removing hair follicles that is constructed in accordance with the invention;

FIG. 11B is a longitudinal sectional view of the punch of FIG. 11A, taken along line 11B-11B in FIG. 11C;

FIG. 11C is a front elevation view, in schematic, of the punch of FIG. 11A,

FIG. 12A is a right front oblique view, in schematic, of a second alternative embodiment of a punch for removing hair follicles that is constructed in accordance with the invention;

FIG. 12B is a longitudinal sectional view of the punch of FIG. 12A, taken along line 12B-12B in FIG. 12C; and

FIG. 12C is a front elevation view, in schematic, of the punch of FIG. 12A.

FIG. 13A shows a longitudinal section view of the punch of FIG. 16, taken along line 13-13 in FIG. 13 with exemplary textured inner surface embodiments.

FIG. 13B shows a side view of the punch.

FIG. 13C shows a front view of the punch.

FIG. 14a exemplarily illustrates a flared embodiment of the hair punch, taken along line 16-16 in FIG. 16.

FIG. 14B shows a side view of an embodiment of the punch.

FIG. 14C shows a front view of an embodiment of the punch.

FIG. 15 exemplarily illustrates resultant a sharp rounded tip embodiment of the hair punch.

FIG. 16 exemplarily illustrates resultant a frustoconical embodiment of the hair punch.

FIG. 16 A [a-c] exemplarily illustrates a textured outer surface for embodiments of the hair punch.

FIG. 16B exemplarily illustrates a textured outer surface for embodiments of the hair punch.

FIG. 16C exemplarily illustrates a textured outer surface for embodiments of the hair punch.

FIG. 17 exemplarily illustrates a longitudinal section view, taken along line 16-16 in FIG. 16 with the general tubular body walls of the follicle punch are reduced.

FIG. 17A exemplarily illustrates a longitudinal section view, taken along line 16-16 in FIG. 16 with the general tubular body walls of the follicle punch are reduced.

FIG. 18 exemplarily illustrates a front view of an embodiment of the punch.

FIG. 18A exemplarily illustrates a front view of an embodiment of the punch.

FIG. 18B exemplarily illustrates a front view of an embodiment of the punch.

FIG. 18C exemplarily illustrates a front view of an embodiment of the punch.

FIG. 19 exemplarily illustrates a longitudinal section view, taken along line 16-16 in FIG. 16 with the general tubular body walls of the follicle punch are reduced.

FIG. 20 exemplarily illustrates a longitudinal section view, taken along line 16-16 in FIG. 16 with the general tubular body walls of the follicle punch are reduced.

FIG. 21 exemplarily illustrates a longitudinal section view, taken along line 16-16 in FIG. 16 with the general tubular body walls of the follicle punch are reduced.

### DETAILED DESCRIPTION OF CERTAIN EMBODIMENTS

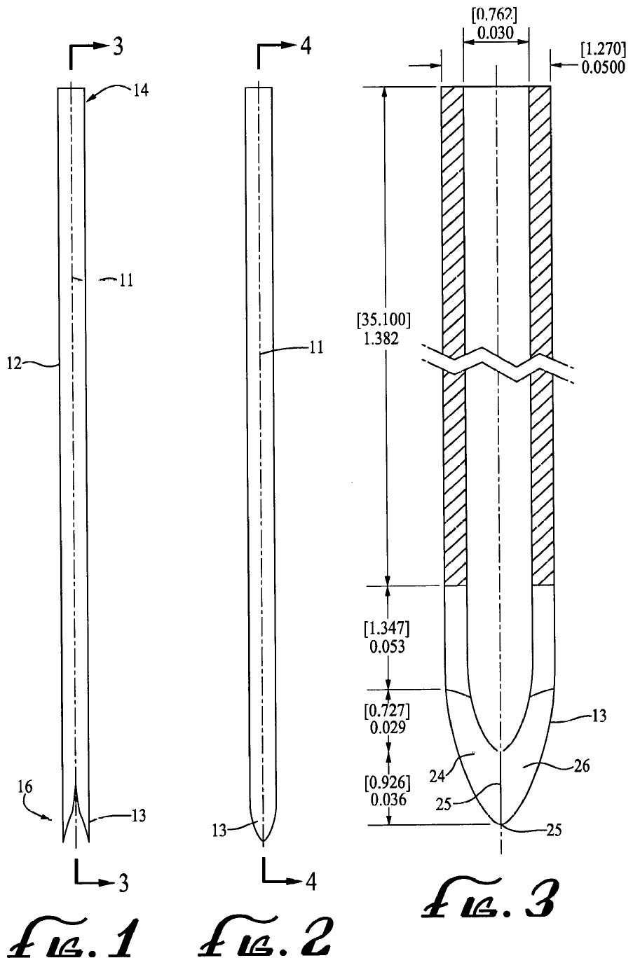

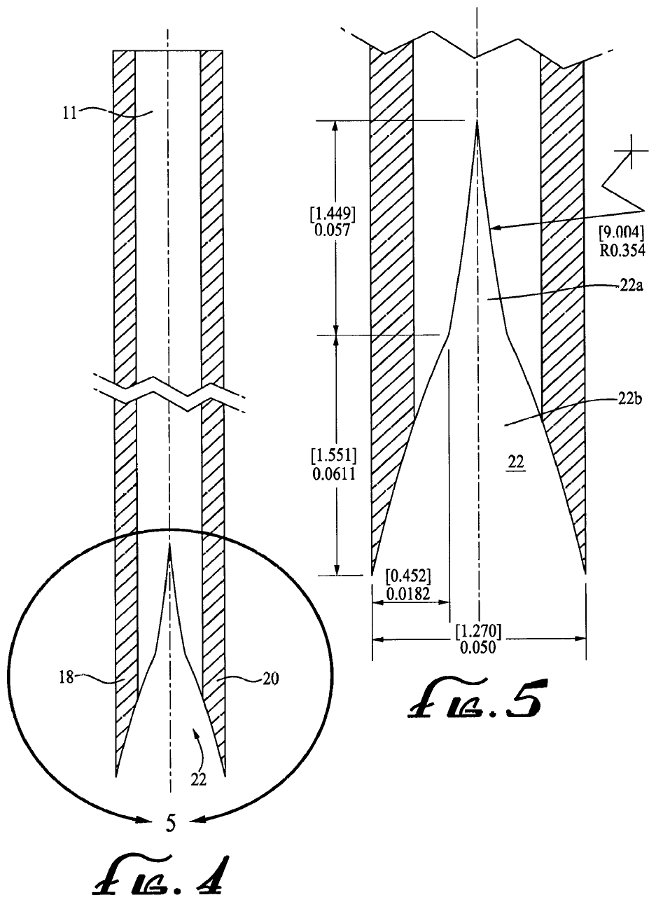

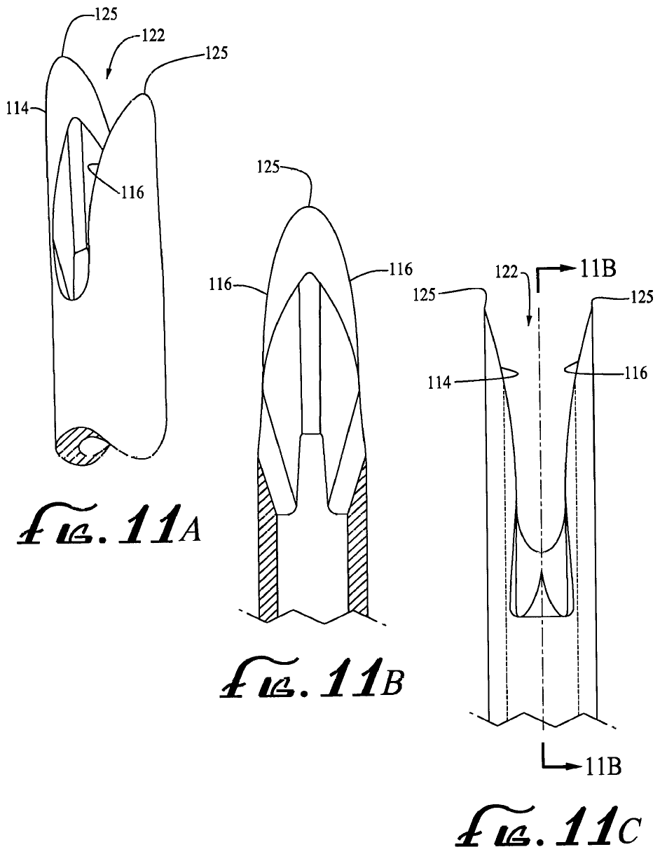

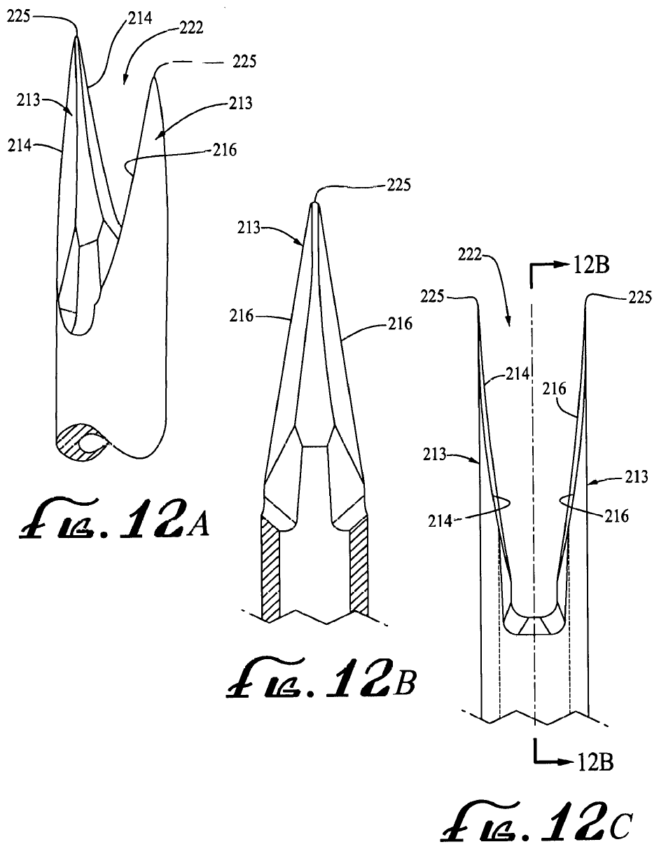

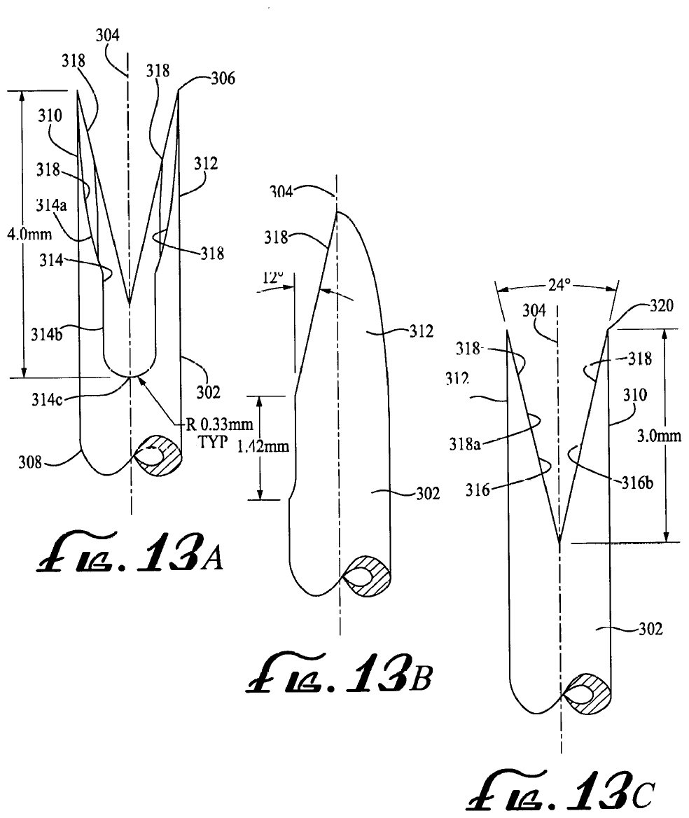

Referring to FIGS. 1-9, a preferred punch for extracting curled follicles is illustrated as comprising a generally tubular body 12 extending from a proximal end 14 to a skin-contacting distal end 16 about a generally central longitudinal axis 11. The currently preferred dimensions of the punch are displayed in the Figures in both inches (unbracketed) and millimeters (bracketed), said currently preferred dimensions being part of this Detailed Description.

The punch's generally tubular body 12 has a distal cutting end region terminating distally in a plurality of distally-extending circumferentially disposed, generally prong-like members 13 carrying distally diverging cutting edges 15 and separated follicle-accommodating slits 22. The currently preferred number of members 13 is two because a pair of such members currently appears to provide the appropriate amount of cutting around the follicle together with sufficient slit width to safely accommodate entry and protection of the follicle during the extraction process.

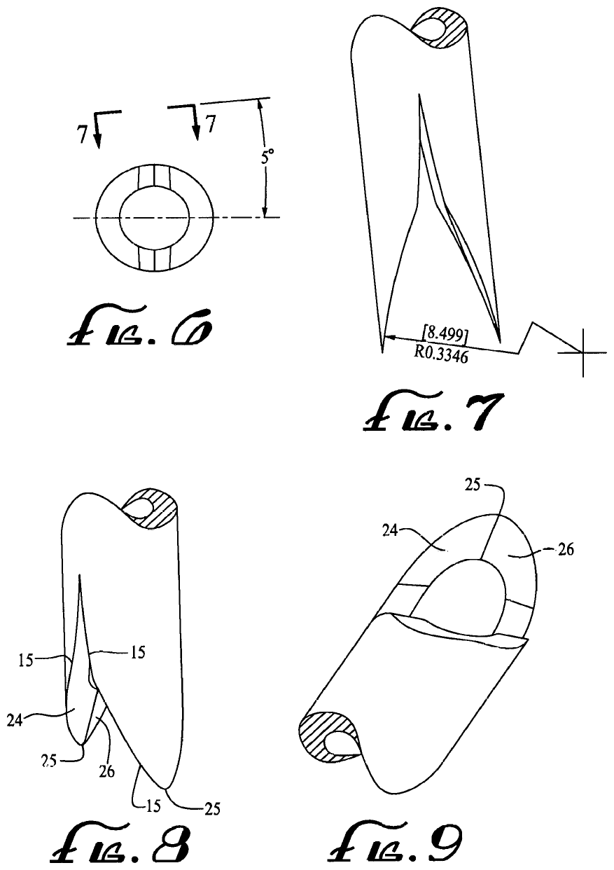

As illustrated in the Figures, each of the preferred prong-like members 13 has a generally convex outer surface and a generally concave inner surface substantially the same as that of the tubular body. As also illustrated in the Figures, each of the prong-like members also has a beveled cutting surface 24, 26 terminating at a cutting edge, with the bevel preferably being on the inside of the punch so that it terminates at a cutting edge on the punch's outer diameter. However, the formation of bevels on the outer surface of the punch is also possible, although not preferred, and is within the scope of the invention. It may be noted that it is currently believed that the cutting edge portion illustrated in FIG. 3 as 1.653 mm in length may be as long as approximately 4 mm or so, and that the follicle-accommodating slit 22 should preferably be approximately 2 mm-4 mm longer than the cutting edge portion.

The bevels 24, 26 are preferably created by grinding cutting edges outwardly from the interiors of the members 13 to produce sharp cutting edges. However, the bevels can also be formed by laser cutting, waterjet or abrasivejet cutting, chemical molding, and/or other manufacturing processes without departing from the scope of the invention.

The bevels 24, 26 preferably interface at an apex of the prong-like member to provide a sharp, point-like, leading tip 25 which makes the initial penetration into the tissue that surrounds the targeted follicle, while the widening, generally semi-elliptical profiles of the prong-like members 13 cut more of the surrounding tissue as the punch is urged distally into the site. The leading tip can alternatively be a sharp rounded tip without departing from the scope of the invention.

The distal end region of the punch may be further provided with generally circumferentially-extending notch having a generally concave shape that generally circumscribes the punch's outer surface. The notch preferably extends 1-2 mm proximally from a location closely adjacent the tip of the punch. The generally concave shape serves two purposes. First, its preferred size and shape results in a wound with everted edges; as the punch enters the tissue surrounding the targeted follicle, the tissue outward of the cut expands against the concavity as it is passed by the cutting edge. When the punch is subsequently withdrawn, the tissue resumes its consequently everted shape. Second, the generally concave shape and preferred sharpening from the inside of the punch results in a cutting force that is outwardly directed away from the follicle and tissue to be extracted, deceasing the risk of damage to the follicle.

Alternatively, the punch can be provided with a flared distal end having a diameter that has a diverging inner diameter and diverging outer diameter along the last 1 mm or so, with the flared end region resulting in a preferred gap of approximately 1.25 mm between opposing tips. Gaps of great or lesser spacing may be utilized as well, depending on the subject's hair and follicle dimensions without departing from the scope of the invention.

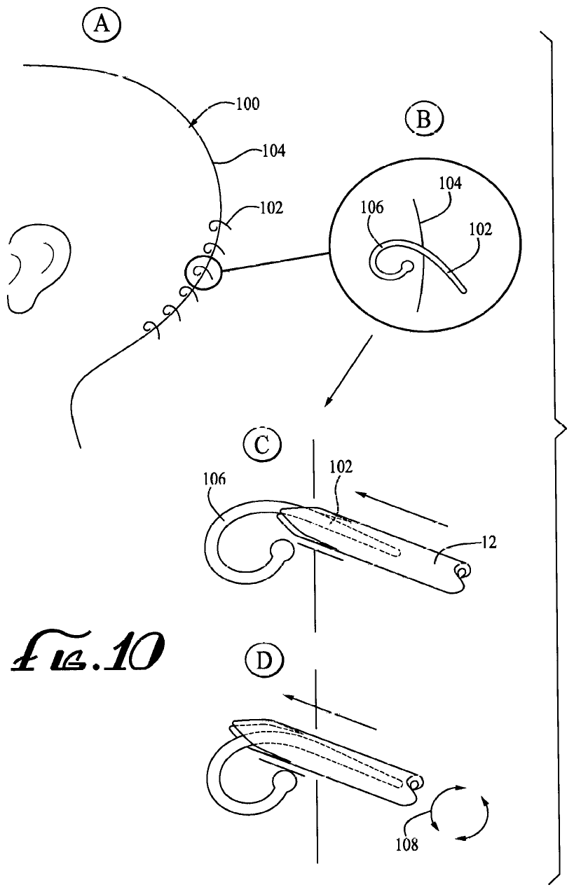

In one preferred configuration, the shape and dimensions of the slit 22 are, as best illustrated in FIGS. 4 and 5, a general inverted “V” profile having a relatively distal segment and a relatively proximal segment 22 a that is more steeply tapered than the distal segment 22 b. The more steeply tapered interior of the relatively proximal segment provides a slit length and width that accommodates the follicle as the punch penetrates the surrounding tissue, in order to spare the follicle from being cut; the less tapered distal segment of the slit results in adequate spacing of the cutting edges of adjacent prong-like members 13 from the follicle's root structure so that the cutting yields a viable implant. Although the same taper could be used for both segments, it is preferable not to do so since a generally uniformly steep taper (such as that of the preferred distal segment) would add unnecessary length to the punch to achieve the needed spacing between the prongs, while a generally uniformly shallow taper (such as that of the preferred distal segment) would fail to provide the slit length needed.

The cutting edge of the punch, which preferably extends from its leading tip to the beginning of the steeply tapered portion of the slit (i.e., the interface of the proximal and distal slit segments), may be smooth or include one or more serrations. If serrations are included, it is currently preferable that there be one or two serrations, with rounded edges, although the use of sharply angled edges would not depart from the scope of the invention.

FIG. 10 schematically illustrates (at “A”) a subject's head 100 having a plurality of hairs 102 protruding from the skin 104. A hair 102 and its curled, subcutaneously-located follicle 106 is schematically illustrated in magnified form at “B”.

As next schematically illustrated with greater magnification at “C”, the preferred punch is inserted into the skin at the donor site in such a way that the hair enters the punch's interior while the follicle 106 passes uncut through the slit 22. As further illustrated at “D”, the punch is advanced past the follicle, which remains undamaged by the cutting edges of the punch by passing through the slit. Once the punch has penetrated sufficiently, it can be partially rotated back and forth if desired, as schematically illustrated by the arrows, resulting in an arcuate cut in the tissue substantially circumscribing the curled follicle, while the follicle itself is spared by its clearance within the slit and isolation from the cutting edges. The intact hair follicle is then removed from the donor site for subsequent transfer to the recipient site.

To penetrate the skin, the punch could be manually pressed proximally by hand. Currently, it is believed that the use of an ultrasonic transducer to apply rapid, incremental, proximally-directed cutting force pulses to the tissue via the punch offers a more precisely controllable methodology for penetrating the tissue while the punch is positioned at the donor site and oriented so as to accommodate the follicle within the slit.

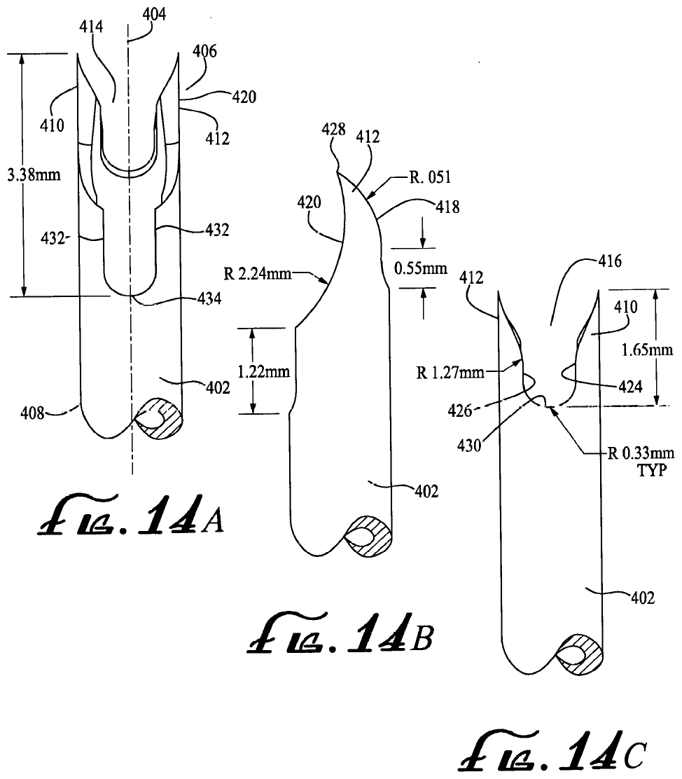

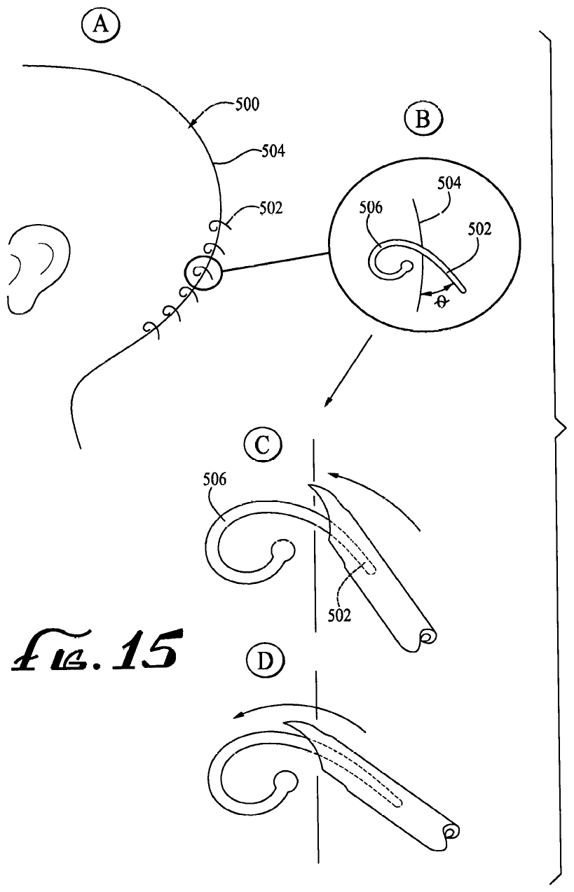

Turning to FIGS. 11A-C, a second currently preferred configuration of a follicle punch is illustrated with its currently preferred dimensions in mm) It should be noted that the interior surfaces of the punch are smooth; the apparent facets illustrated in FIGS. 11A and 11B are computer-generated “tangent” lines connoting a change in surface direction only.

The punch illustrated in FIGS. 11A-C comprises a pair of distally-extending circumferentially disposed, generally prong-like members 113 carrying distally diverging cutting edges 114, 116 and separated by a generally U-shaped follicle-accommodating slit 122. The cutting edge of each prong-like member is again preferably formed from the inside of the punch by grinding cutting edges outwardly from the interior region of the members. However, as noted earlier, the cutting edges can also be formed by laser cutting, waterjet or abrasivejet cutting, chemical molding, and/or other manufacturing processed without departing from the scope of the invention. The leading tips 125 of the punch illustrated in FIGS. 11A-C are sharp rounded tips that make the initial penetration into the skin and tissue surrounding the targeted follicle.

To minimize the risk of the follicle being cut during the extraction process, the formation of the cutting edges may be limited to the first 0.060 inches (1.52 mm) or so from the distal tip 125 of the punch, so that the cutting edges pass the follicle during insertion of the punch at the donor site and any subsequent contact between the punch and follicle is not with a cutting edge. The cutting edge may however extend the entire length, or a different length, of the slits.

The gap between the prong-like members of the punch illustrated in FIGS. 11A-C is preferably 0.02-0.03 inches (0.51-0.76 mm) wide. It preferably extends proximally from the distal tip of the punch for about 0.12 to 0.16 inches (3.05-4.06 mm)

Turning to FIG. 12A-C, another preferred variation of the punch is illustrated, wherein the punch comprises a pair of distally-extending circumferentially disposed generally prong-like members 213 that carry distally-diverging cutting edges 214, 216 separated by a generally U-shaped follicle-accommodating slit. The leading tips 225 of the members 213 are sharp pointed tips. Each cutting edge 214, 216 is preferably formed from the inside of the punch by grinding cutting edges outwardly from the interior region of the prong-like members. However, as noted earlier, the cutting edges can also be formed by laser cutting, waterjet or abrasivejet cutting, chemical molding, and/or other manufacturing processed without departing from the scope of the invention. To minimize the risk of the follicle being cut during the extraction process, the formation of the cutting edges may be limited to the first 0.060 inches (1.52 mm) or so from the distal tip of the punch, so that the cutting edges pass the follicle during insertion of the punch at the donor site and any subsequent contact between the punch and follicle is not with a cutting edge. The cutting edge may however extend the entire length, or a different length, of the gap.

The gap between the prong-like members of the punch illustrated in FIGS. 12A-C is preferably 0.03 inches (0.076 mm) wide, and preferably extends proximally from the distal tip of the punch for about 0.16 inches (4.06 mm)

As with FIGS. 11A-B, it should be noted that the interior surfaces of the punch illustrated in FIGS. 12A-B are smooth, and that the apparent facets are computer-generated “tangent” lines connoting a change in surface direction only.

In some embodiments, as shown in FIG. 13 and FIGS. 13A-C, the device can be altered with fine adjustments. There is increased resistance as the tool is advanced into the skin because of the expanding size of the follicle and tissue to be removed. Consequently, measures can be taken to reduce the resistance and enhance tissue penetration. The inner surface of the general tubular body of the punch 12, in some embodiments, may be modified by one he following means including: a texture, a patterned projections and a patterned indentations. Patterned or un-patterned inner surfaces of the general tubular body of the punch 12 be arranged within the distal end region 16, up to or including prong members 13. In some embodiments, the texture of the inner surface of the distal end region 16 is at least one member of a texture set including, by way of non-limiting examples, the following: grooving, hexing, ridging, serrating, etching, knurling, brushing, ribbing, stippling, and coble-stoning.

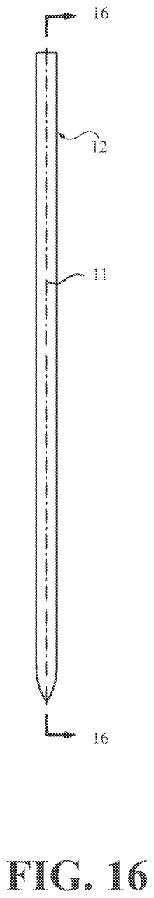

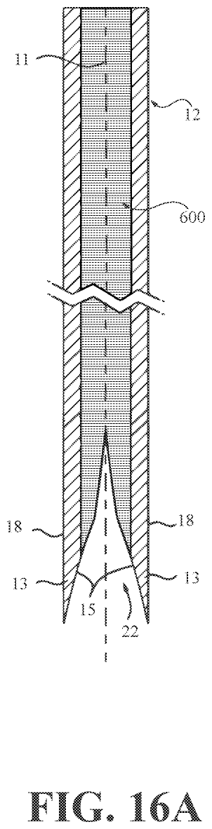

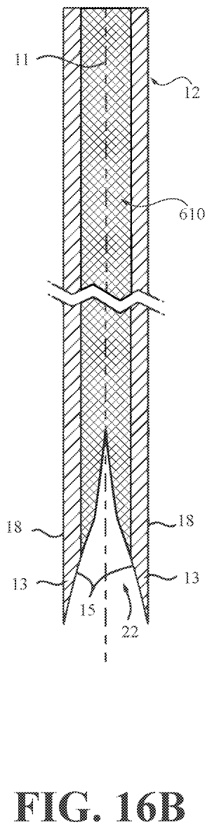

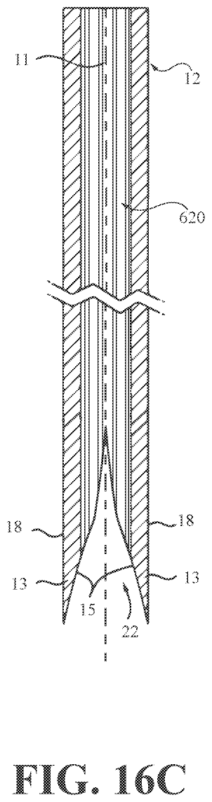

In some embodiments, as shown in FIG. 13C, longitudinal grooves 620 aid or guide penetration when the punch 12 is used in a plunging manner, rather than a rotary manner. Longitudinal grooves 620 could be of any length or depth relative to the overall length of the generally tubular body of the punch 12. Additionally, measures can be taken to increase resistance and decrease tissue penetration. Horizontal grooves 600 patterns, as shown in FIG. 16A, or diamond etching 610, as shown in FIG. 16B, patterns may be incorporated into or protrude from the inner surface of the generally tubular body of the punch 12. Horizontal grooves 600 patterns or diamond etching 610 patterns could incorporated into or protrude from the inner surface at any length or depth of the generally tubular body relative to the overall length the punch 12.

As shown in FIGS. 14 and 14A, in some embodiments the punch 12 may have prong member 13 that flare outward 630 from central axis 11. The distance between distally diverging cutting edges 15 is increasing as the prong members 13 come to a point at the end of the distal end region 16.

As shown in FIG. 15 and FIG. 16, in some embodiments, the prong members 13, the entire length of the distal end region 16 or a partial length of the distal end region 16 may be a frustoconical design 640 and have multiple flat sides. FIG. 15 illustrates a frustoconical design in a sharp rounded tip embodiment of the hair punch. FIG. 16 exemplary illustrates a frustoconical embodiment of the hair punch coming to a sharp point. The frustoconical design 640, of some embodiments, may have at least three flat sides and allow the user the option to have a similar circular tubular body for the punch 12 by way of multiple flat frustoconical sides.

As shown in FIGS. 15A-C and FIG. 16A-C, in some embodiments, the outer surface 18 of the general tubular body of the distal end region 16 of the punch 12, up to or including prong members 13, can be enhanced to increase the ability of the punch to separate the scored hair follicles form it's respective anchoring tissue by several features including: texture, patterned projections and patterned indentations. These enhancements can manipulate friction between the punch 12 and the patient's anchoring tissue and increase the ability of the user to grasp the punch 12. In some embodiments, the texture of the outer surface 18 of the distal end region 16 is at least one member of a texture set including, by way of non-limiting examples, the following: grooving, hexing, ridging, serrating, etching 670, knurling, brushing, ribbing, stippling 650, and coble-stoning.

Finally, as shown in FIGS. 15B and 16B, horizontal grooves 660 that are complete or partially disposed in outer surface 18 of the distal end region 16 of the punch 12, up to or including prong members 13, that exert a ribbed like effect to enhance forward advancement when punch is used in a plunging manner. In some embodiments, the texture, patterned projections or patterned indentations on the outer surface 18 are located in a complete or partial part of the distal end region 16 of the punch up to or including prong members 13. Additionally, these grooves in some embodiments are located in front of the distal end region 16 of the punch up to or including prong members 13 to have a pulling effect on it to aid the punch's 12 ability to penetrate the patient's tissue despite its increasing width.

In some embodiments, a suction device could be either in front of, within or behind the distal end region 16 of the punch 12, up to or including prong members 13. The suction effect could be increased by increasing an internal diameter of the general tubular body of the punch 12 to achieve a Bernoulli effect either in front, within or behind the distal segment of the punch up to or including prong segments. Mechanical movement of the punch 12 to drive the into the tissue in some embodiments can be adjusted by a driver that could be rotary, oscillatory, vibratory, ultrasonic, pneumatic means. Friction between tissue and distal end region 16 of the punch 12, up to or including prong members 13, could be manipulated by polishing, lubrication, manipulation of electrostatic properties, and temperature control of the generally tubular member of punch 12.

In some embodiments, the process can be enhanced by the effect of a fluid egressing mechanism which creates a fluid plane interface between punch cutting edges 15 and the skin or tissue of the patient. The purpose of the fluid egressing mechanism is to wet the grafts during scoring as well as to flush out grafts or debris impacted inside the punch or generally tubular body.

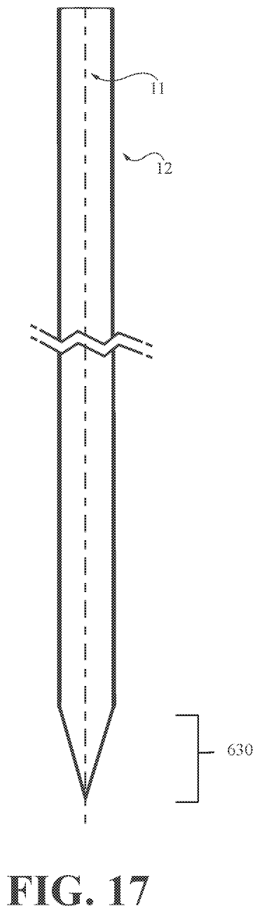

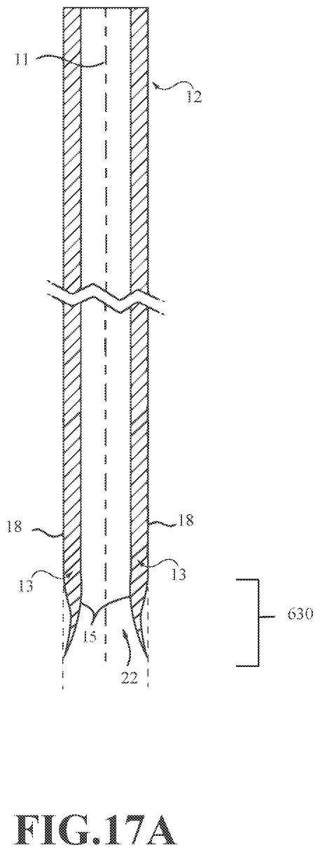

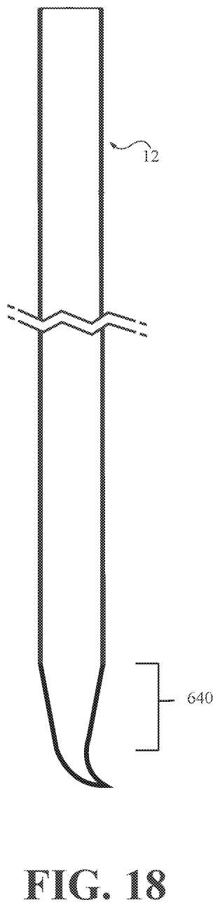

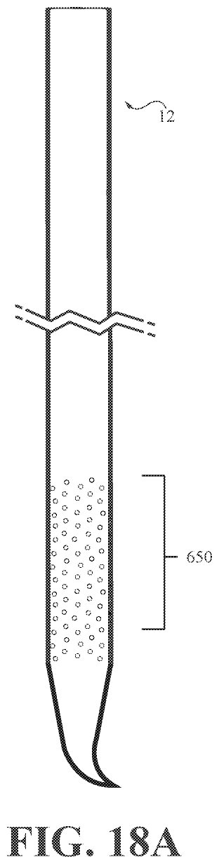

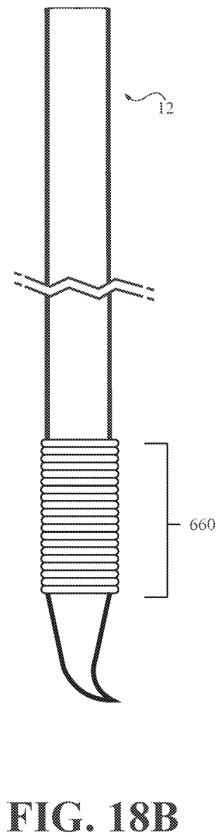

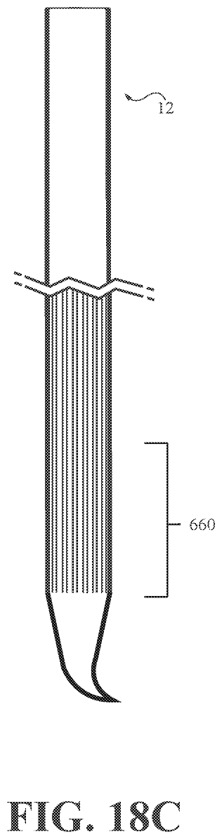

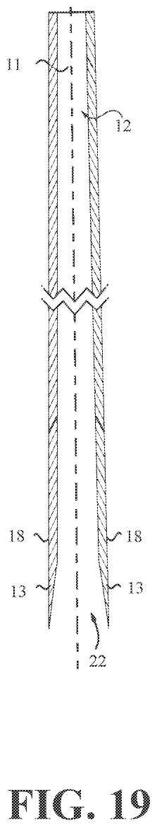

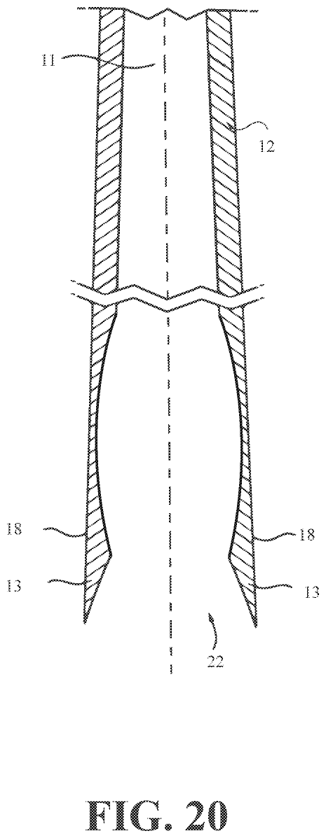

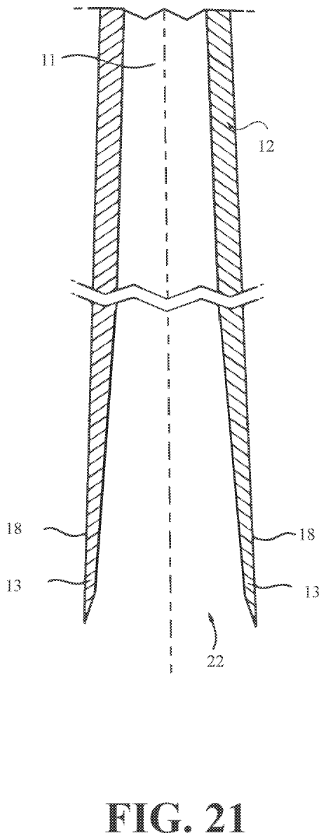

As shown in FIG. 17-19, in some embodiments, the internal diameter the generally tubular body of the punch 12 from the distal end region 16 of the punch, up to or including prong members 13, can be increased by etching or removal of the inner surface of the generally tubular body by removing the inner 25-75% of the tube wall thickness. FIG. 19 illustrates a uniform reduction of the internal diameter of the generally tubular body. FIG. 19 illustrates a uniform reduction of the generally tubular body. FIG. 20 illustrates a concave reduction of the internal diameter of the generally tubular body. FIG. 21 illustrates a slanted reduction of the internal diameter of the generally tubular body.

Regardless of the specific version of follicle punch utilized, the inclusion of an ultrasonic transducer coupled to the punch and selectively operable to enhance the cutting operation is desirable. The transducer is mounted within a handpiece to which the punch is attached in a manner analogous to the transducer, handpiece and scaler tip of an ultrasonic dental scaler. The punch may be further mounted for reciprocating pivoting movement within the handpiece so as to move in such manner with or without ultrasonic vibratory movement. Likewise, the configuration may be such that ultrasonic vibratory movement can be generated with or without the pivoting movement.

In practice, it has been found that an adjustable degree of longitudinally reciprocating ultrasonic movement is desirable in that the appropriate degree of movement is a function of the subject's skin thickness and tissue, with higher settings being suitable when cutting through thicker skin or scar tissue for example. The use of the ultrasonic movement permits the surgeon or other operator of the equipment to better use his/her “fine motor” muscle movement to more precisely make the required incisions with greater sensitivity and finesse.

Although the present invention and its advantages have been described in detail, it should be understood that various changes, substitutions and alterations can be made herein without departing from the spirit and scope of the invention as will be defined by appended claims.

What is claimed is:

1. A follicle punch comprising:
a generally tubular body disposed about a generally longitudinal axis between distal and proximal ends, and having
an inner surface, having an internal diameter and an outer surface, having an outer diameter; and
a pair of distally-extending, circumferentially disposed, prong-like members carrying distally diverging cutting edges separated by follicle-accommodating slits.

2. The follicle punch of claim 1, wherein the wherein the prong-like members are arranged to terminate distally in respective leading tips that make an initial penetration into tissue surrounding a follicle, and a distal end region of the follicle punch includes a circumferentially-extending notch having a concave shape that circumscribes the outer surface of the follicle punch.

3. The follicle punch of claim 2, wherein the circumferentially-extending notch extends distally 1-2 mm from the leading tips of the prong-like members.

4. The follicle punch of claim 2, wherein the follicle-accommodating slits each have an inverted “V” profile.

5. The follicle punch of claim 4, wherein the inverted “V” profile comprises a relatively proximal segment and a distal segment that is more steeply tapered than a distal cutting end region.

6. The follicle punch of claim 4, wherein the prong-like members are flared to direct the leading tips away from the generally longitudinal axis by an angle between 1 and 60 degrees.

7. The follicle punch of claim 4, wherein the prong-like members are flared to direct the leading tips towards from the generally longitudinal axis by an angle of between 1 and 60 degrees.

8. The follicle punch of claim 4, wherein one or more of the distally diverging cutting edges is blunt.

9. The follicle punch of claim 4, wherein one or more of the distally diverging cutting edges is sharp.

10. The follicle punch of claim 4, wherein the circumferentially-extending notch come to an end distally at differential levels that range from 0.25 to 1.5 mm.

11. The follicle punch of claim 1, wherein longitudinal grooves are arranged on the prong-like members.

12. The follicle punch of claim 11, wherein the longitudinal grooves are arranged on the inner surface of the follicle punch.

13. The follicle punch of claim 11, wherein the longitudinal grooves are arranged on the outer surface of the follicle punch.

14. The follicle punch of claim 1, wherein the prong-like members are textured by one member of a texture set consisting of: grooving, hexing, ridging, serrating, etching, knurling, brushing, ribbing, stippling, and coble-stoning.

15. The follicle punch of claim 1, wherein the inner surface is textured by one member of a texture set consisting of: grooving, hexing, ridging, serrating, etching, knurling, brushing, ribbing, stippling, and coble-stoning.

16. The follicle punch of claim 1, wherein the outer surface is textured by one member of a texture set consisting of: grooving, hexing, ridging, serrating, etching, knurling, brushing, ribbing, stippling, and coble-stoning.

17. The follicle punch of claim 2, wherein the leading tips of the distally diverging cutting edges are rounded.

18. The follicle punch of claim 1, wherein the generally tubular body is frustoconical.

19. The follicle punch of claim 1, wherein the distally diverging cutting edges are made by one member of a manufacturing set consisting of: laser cutting, electrical discharge machining, waterjet cutting, abrasive jet cutting, and chemical molding.

20. The follicle punch of claim 1, wherein the follicle punch further comprises a fluid egressing mechanism, wherein the fluid egressing mechanism creates a fluid plane between the distally diverging cutting edges and a tissue of a patient to assist in cutting and flushing within the generally tubular body.

21. The follicle punch of claim 1, wherein the generally tubular body further comprises an increased inner diameter consisting of at least 125% of the internal diameter with a reduced wall thickness.

22. The follicle punch of claim 21, wherein the reduced wall thickness is formed by removing material by one member of a manufacturing set consisting of: laser cutting, electrical discharge machining, waterjet cutting, abrasive jet cutting and chemical molding.
